(19) United States
(12) Patent Application Publication
(10) Pub. No.: US 2002/0103165 A1
(43) Pub. Date: Aug. 1, 2002

(54) Engineered spray-dried lipid-based microparticles for cellular targeting

(75) Inventors: Adrian Bot, San Diego, CA (US); Luis Dellamary, San Marcos, CA (US); Dan Smith, San Diego, CA (US); Catherine M. Woods, La Jolla, CA (US)

Correspondence Address: Lisa A Haile, Gray Cary Ware & Freidenrich LLP, Suite 1600, 4365 Executive Drive, San Diego, CA, 92121-2189, US

(73) Assignee: ALLIANCE PHARMACEUTICAL CORP.,

(21) Appl. No.: 09/515,359

(22) Filed: Feb. 29, 2000

(51) Int. Cl. A61K 31/665

(52) U.S. Cl. 514/100

(57) ABSTRACT

In accordance with the present invention there are provided novel Spray-Dried Lipid Microparticles (SDLM) that are comprised of lipid, a ligand and agent. The ligand is specific for a cell surface receptor, thereby enabling targeting of the agent to cells bearing receptors specific for the targeting ligand. In invention embodiments wherein the receptor internalizes upon ligand binding, there are provided compositions and methods for the introduction of agent into specifically targeted cells. In a particular embodiment, the ligand is specific for antigen presenting cells (APC) and the agent is a defined antigen. In this manner, immune responses can be induced against specific antigens, including those which are normally not very antigenic due to the fact that they are inefficiently internalized by APC.

### FIELD OF THE INVENTION

The present invention relates generally to the field of microparticles and more specifically to lipid-based microparticles and their use for introducing agents into a cell.

### BACKGROUND OF THE INVENTION

Vertebrates possess the ability to mount an immune response as a defense against pathogens from the environment as well as against aberrant cells, such as tumor cells, which develop internally. This can take the form of innate immunity, which is mediated by NK cells, neutrophils and cells of the monocyte/macrophage lineage, or the form of acquired or active immunity against specific antigens mediated by lymphocytes. Active immune responses can be further subdivided into two arms, the humoral response which entails the production of specific antibodies that serve to neutralize antigens exposed to the systemic circulation and aid in their uptake by professional phagocytic cells, and the cellular arm which is required for recognition of infected or aberrant cells within the body.

In both cases the specific response is regulated by the intracellular processing and recognition of the antigen by effector T-cells. Mature cytolytic T lymphocytes (CTLs) or T helper cells (Th), remain in a resting state unless they encounter antigens that their receptors can recognize in the context of MHC class I or II molecules. Upon encountering the specific antigens, the T-cells proliferate and perform effector functions, the result of which is elimination of the reactive antigens. When the antigen is synthesized in the cell, it is subsequently processed through the cytoplasmic route and the resultant peptides are bound to nascent MHC class I molecules which facilitate appropriate presentation to effector T-cells. MHC class I presentation favors recognition by cytotoxic T lymphocytes (CTLs) that carry the CD8 ligand. In contrast, intracellular processing via the endocytic route results in presentation on MHC class II molecules which favors T helper responses involved in stimulation of the humoral arm. The goal of vaccination is to prime both responses and generate memory T cells, such that the immune system is primed to react to a pathogenic infection. Engagement of both the humoral and cellular immune responses leads to broad based immunity and is the preferred goal for intracellular pathogens.

Activation of the T cells entails the generation of a cascade of chemical signals, primarily cytokines, that result in either direct action or the stimulation of other cells of the immune system to act. In the case of activation by class I MHC-antigen, CTLs proliferate and act to destroy infected cells presenting that given antigen. Killing an infected cell prevents the virus from proliferating and makes it accessible to neutralizing antibodies, and hence permitting elimination of the virus. In contrast, activation of Th cells by class II MHC-antigen complexes does not destroy the antigen presenting cell (which is part of the host's defense system) but rather stimulates the Th cell to proliferate and generate signals (again primarily cytokines) that affect various cells. Among other consequences, the signaling leads to B cell stimulation, macrophage activation, CTL differentiation and promotion of inflammation. This concerted response is relatively specific and is usually directed to foreign elements bearing the peptide presented by the class II MHC system.

When functioning properly, the immune response is surprisingly effective at eliminating microscopic pathogens and, to a lesser extent, neoplastic cells. However, among the many difficulties that exist in marshalling the body's defenses against pathogens, neoplastic disease related cells, and the like, is inducing an appropriate immune response. One of the barriers to the induction of such a response is the fact that certain extracellular antigens do not readily enter immune system cells such as macrophages and immature dendritic cells that process antigen for cell surface display via either MHC class I or class II routes (e.g., antigen presenting cells (APC)). Thus, antigens are not presented to other immune system cells (e.g., Th cells and CTLs) in a manner that readily provokes an immune response.

### BRIEF DESCRIPTION OF THE INVENTION

In accordance with the present invention, there are provided Spray-Dried Lipid-based Microparticles (SDLM) that are engineered to be able to specifically target an agent to an internalizable cell surface receptor by incorporating a specific ligand within the microparticle matrix. By employing invention ligand-SDLM, it is possible to direct a specific agent to a specific cell type. Invention ligand-SDLM are thereby useful for inducing an immune response, targeted cell killing, and the like.

In one embodiment of the invention, there is provided ligand-containing Spray-Dried Lipid Microparticle composition. Invention ligand-SDLM include one or more phospholipids, one or more therapeutic or biologically active agent, and at least one ligand that binds to a cell surface receptor.

In another embodiment of the present invention, there are provided ligand-SDLM useful for inducing or enhancing an immune response. In one aspect, the agent can be an antigen or a nucleic acid encoding an antigen.

In another embodiment of the present invention, there are provided methods for the introduction of a therapeutic or biologically active agent into a cell of a subject by administering to a subject a ligand-SDLM composition of the invention. The ligand-SDLM composition includes one or more phospholipids, a therapeutic or biologically active agent, and at least one ligand that binds to a cell surface receptor.

In yet another embodiment of the present invention, there are provided methods for inducing or enhancing an immune response in a subject by administering an effective amount of ligand-SDLM containing an agent that is antigenic or encodes one or more antigens.

In a further embodiment of the invention, there are provided methods for suppressing the activity of pathogenic T cells by administering to a subject an effective amount of ligand-SDLM as described herein. Pathogenic T cells mediate autoimmune diseases (e.g., diabetes type 1, multiple scleorosis) or inflammatory diseases (e.g., exacerbated delayed hypersensitivity conditions) by virtue of producing pro-inflammatory cytokines (IFN-gamma, TNF-α) and/or directly lyse host cells via perforin or Fas-mediated pathways, for example.

In still another embodiment of the invention, there are provided methods to enhance the activity of T suppressor cells by administering to a subject an effective amount of ligand-SDLM.

In a related embodiment, there are provided methods to induce the production of suppressor cytokines (e.g., IL-4, IL-10, TGF-β, IL-13) by antigen presenting cells by administering to a subject an effective amount of ligand-SDLM.

In another embodiment of the present invention, there are provided methods of selectively killing or inhibiting the growth of neoplastic cells by administering to a subject an effective amount of ligand-SDLM.

### BRIEF DESCRIPTION OF THE FIGURES

FIG. 1 graphically shows that the internalization of microparticles is dependent on the presence and amount of IgG ligand incorporated in the microparticles.

FIG. 2 shows co-internalization of ligand and lipid matrix into phagocytes.

FIG. 3 is a histogram showing that internalization of SDLM is dependent on ligand (IgG) being incorporated into the SDLM rather than simply being present in the media at the time SDLM are added.

FIG. 4 is a histogram showing that excess free IgG can competitively inhibit internalization of SDLM with IgG ligand, thereby indicating that internalization of IgG-containing SDLM is IgG receptor specific.

FIG. 5 is a histogram showing that internalization of SDLM is an active process requiring living cells at a permissive temperature.

FIG. 6 is a histogram showing that high molecular weight carbohydrate is effective as a stability-conferring excipient when compared to low molecular weight carbohydrate, and that the effect does not vitiate the need for ligand (IgG).

FIG. 7 is a histogram showing that ligand-engineered SDLM mediate more effective internalization of the ligand into phagocytes, as measured by flowcytometry analysis.

FIG. 8A is a histogram showing the release kinetics of antigen from SDLM containing ligand and antigen.

FIG. 8B is a histogram showing the release kinetics of ligand from SDLM containing ligand alone.

FIG. 9 shows the activation of specific T cells by alveolor phagocytes pulsed with antigen-SDLM.

FIG. 10 is a histogram showing the presentation of viral antigen by plastic adherent (10A) or non-adherent (10B) lung phagocytes pulsed with ligand-SDLM.

FIG. 11 is a histogram showing the effect of ligand-engineering of SDLM on presentation of viral antigen by splenic phagocytes.

FIG. 12 shows the dependency of antigen presentation on ligand-mediated internalization of SDLM.

### DETAILED DESCRIPTION OF THE INVENTION

In order to specifically direct agents to certain cells, the present invention takes advantage of the fact that many cells have internalizable cell surface receptors (ICSR). One means for facilitating the internalization of an agent by a cell is to induce binding of the agent to an ICSR. The present invention provides a vehicle for delivering an agent into a cell package that is biocompatible and which is targetable to specific ICSR. The novel vehicles described herein are termed Spray-Dried Lipid-based Microparticles (SDLM) and are uniquely engineered to target an agent to an ICSR by incorporating a specific ligand within the SDLM (also referred to as ligand-SDLM). Invention ligand-SDLM are formulated from one or more phospholipids, one or more therapeutic or biologically active agents, and at least one ligand that binds to a cell surface receptor. In a preferred embodiment, the ligand and agent are physically coupled (e.g., ionically bonded, covalently bonded, or the like). In this manner, the site of ligand binding determines the site to which agent is delivered. Invention ligand-SDLM, and methods employing same, are particularly useful for the introduction of antigen into antigen presenting cells (APC), thereby inducing MHC class I and/or class II-mediated responses. Moreover, the present invention is useful for presenting any number of therapeutic or biologically active agents to cells for internalization via ICSR. For example, as further described herein, the present invention is useful for directing therapeutic genes to cells having specific ICSR.

Ligand-SDLM of the present invention can be viewed as packaging and delivery systems. The lipid component of the SDLM operates as a biocompatible container that, subsequent to contact with an aqueous environment (e.g., blood, or the like), undergoes restructuring leading to the release of formulated compounds (e.g., ligand-agent complex). Any phospholipid suitable for the generation of spray-dried microparticles can be employed in the practice of the present invention. Specific phospholipids contemplated for use in the practice of the present invention include, but are not limited to, phosphatidylcholine and/or derivatives thereof such as dipalmitoylphosphatidylcholine; dystearylphosphatidylcholine; dimiristoylphosphatidylcholine, and the like, and combinations of two or more thereof.

Within the phospholipid component of invention ligand-SDLM are the targeting ligand and the agent. The ligand is the specificity-conferring component of the ligand-SDLM. Targeting is accomplished by employing a ligand for a cell surface receptor (CSR) that is specific for a certain cell type and/or a desired response.

Any ligand recognized by a CSR can be employed in the practice of the present invention. Specific non-limiting examples of CSR for which ligands can be employed include Fc receptor (FcR) e.g., FcγR I, II or III, whether expressed on multiple antigen presenting cells, monocytes, macrophages, dendritic cells, B cells, M cells, or the like), mannose receptor, scavenger receptor, viral receptor, M-cell receptor, ganglioside GM1 receptor, transferrin receptor, complement receptors, surface immunoglobulins on B cells, T cell receptors on T cells, peptidoglycan receptor, lipopolysaccharide (LPS) receptor, glycolipid receptor, and the like. In a preferred embodiment of the present invention, CSR to which the ligand component of invention ligand-SDLM bind are coupled with a signal cascade leading to the endocytosis/phagocytosis of the receptor upon binding of ligand to the receptor (e.g., Fe receptor, hormone receptor, and the like). Those of skill in the art can readily identify such signal cascade-coupled receptors (e.g., a cascade leading to cytokine production and/or phagocytosis), and target them according to a desired outcome (e.g., therapeutic, diagnostic,).

Accordingly, ligands that can be employed in the practice of the present invention include those that bind to receptors coupled with signal transduction cascades and/or are internalizable. Specific ligands include, but are not limited to, the Fc portion of immunoglobulins, whether polyclonal or monoclonal, syngeneic, allogeneic, or xenogeneic, for example. Antibodies or portions thereof that bind to cellular receptors including FcR, mannose receptors, scavenger receptors, viral receptors (e.g., ICAM-1 for rhinovirus), M-cell receptors (e.g., ganglioside GM1 receptor for cholera toxin, and the like), transferrin receptor, and the like can be employed as ligand. Any portion of a protein or other molecule that is specific for a CSR may also be employed in the practice of the present invention. For example, while a complete antibody would bind to an Fc receptor, only the Fc portion is actually required for the binding to occur. Thus, suitable ligands include recombinant polypeptides that include, for example, an Fc portion of an antibody, the portion of an LPS molecule that binds its cognate CSR, and the like. Recombinant antibody-epitope constructs may be used, for example, including ligand and agent in the same molecule.

In one embodiment of the present invention, there are provided ligand- SDLM compositions and methods of use thereof for inducing or enhancing an immune response in a subject. Cell surface receptors that may be targeted for ligands of invention ligand-SDLM used to induce or enhance an immune response include FcR, (e.g., FcγR I, II or III, whether expressed on multiple antigen presenting cells, monocytes, macrophages, dendritic cells, B cells, M cells, or the like); other CSR on APC, including, but not limited to, mannose receptor, transferrin receptor, and the like, as well as other receptors that are coupled with a signaling cascade leading to cytokine production and phagocytosis). A preferred targeted receptor is an FcγR II/III, preferably expressed on an APC.

In another embodiment of the present invention, there are provided ligand-SDLM compositions and methods of use thereof for suppressing the activity of pathogenic T cells, enhancing the activity of T suppressor cells, or inducing the production of suppressor cytokines by APC. Such receptors may include any of the CSR described herein, provided that they are present on APC. Accordingly, ligands described herein that are present on an APC may be used in invention ligand-SDLM employed for suppressing the activity of pathogenic T cells, enhancing the activity of T suppressor cells or inducing the production of suppressor cytokines by APC.

Agents contemplated for use in the practice of the present invention include any protein, nucleic acid molecule, small molecule or other compound which may be chosen for cell-specific targeting, whether to induce or suppress (i.e., modulate) an immune response, kill or inhibit growth of the target cell, express or inhibit the expression of a gene in a target cell, or the like. Agents include, but are not limited to, polypeptides, peptides, peptidomimetics, nucleic acid molecules (e.g., DNA, antisense) small molecules, and the like. Agents may be biologically active compounds and/or therapeutic compounds. Types of agents contemplated for use in the practice of the present invention include antigens, toxins, nucleic acids, detectable markers (e.g., radiolabels), and the like.

As used herein, “polypeptide” is understood to be any translation product of a polynucleotide regardless of size, and whether glycosylated or not. Therapeutic polypeptides contemplated for use in the practice of the present invention include polypeptides that induce an immune response in a subject. For example, immunomodulatory agents and other biological response modifiers can be administered for incorporation by a cell. As used herein, the term “biological response modifiers” encompasses substances which are involved in modifying the immune response, without engaging specific antigen receptors on B and T cells. Examples of immune response modifiers include such compounds as lymphokines/cytokines, and the like. Lymphokines and cytokines contemplated for administration as agent in accordance with invention methods include, for example, tumor necrosis factor, interleukins (e.g., IL-1, IL-2, IL-3, IL-4, IL-10, IL-12), lymphotoxin, macrophage activating factor, migration inhibition factor, colony stimulating factor, alpha-interferon, beta-interferon, gamma-interferon, and their subtypes, costimulatory or inhibitory molecules such as B7.1, B7.2, CD40, CTLA-4 and FasL, and the like, or antibodies that bind to cytokine receptors or co-stimulatory molecules.

Antigenic agents contain at least one epitope and include polypeptides, peptides, and inactivated pathogens. As a practical matter, any antigen may be employed so long as it is capable of inducing an immune response, e.g., by either an MHC class I or class II-mediated pathway.

Antigens may also be tumor-derived or pathogen-derived. Pathogen-derived antigens may be either a portion of the pathogen (e.g., protein, LPS, or the like) or an entire inactivated pathogen, for example. Pathogens from which antigens may be derived include virus, bacteria, yeast, fungus, protozoa, and the like. Any pathogenic virus may be the source of a pathogenic viral-derived antigen. Such viruses include whole killed virus and subunits thereof (including recombinant subunits) of influenza virus (e.g., orthomyxoviruses), respiratory syncitial virus (e.g., F or G antigens), papiloma virus, herpes simplex I or II virus, cytomegalovirus, Epstein Barr virus, measles virus, rhinovirus, poliovirus, rotavirus, varicella-virus, HIV (e.g., gp 120), and the like.

Agents may be chosen for particular applications. For example, in one embodiment of the present invention, an agent is selected for use with an invention ligand-SDLM to induce or enhance a protective immune response, cellular or humoral. Agents useful for this embodiment include antigens derived from pathogens including virus, bacteria, yeast, fungus, protozoa, and the like, as further described below.

Any pathogenic bacteria may be the source of a pathogenic bacterial-derived antigen. Such bacteria include Staphylococcus, Clostridium, Streptococcus, Enterococcus, Diplococcus, Hemophilus, Neisseria, Erysipelothricosis, Identifyeria, Bacillus, Salmonella, Shigella, Escherichia, Klebsiella, Enterobacter, Serratia, Proteus, Morganella, Providencia, Yersinia, Camphylobacter, Mycobacteria, Mycoplasma, and the like.

Any pathogenic protozoa may be the source of a pathogenic protozoa-derived antigen. Such protozoa include Plasmodium, Trypanosoma, Microfilariae, Leishmania, Giardia, Entamoeba, Schistosoma, Cryptosporidium Entamoeba, Pneumocystis, Toxoplasma, and the like.

Any pathogenic fungus may be the source of a pathogenic fungus-derived antigen. Such fungus include Histoplasma, Coccidioides, Cryptococcus, Blastocyces, Paracoccidioides, Candida, Aspergillus, Nocardia, Sporothrix, Rhizopus, Absidia, Mucor, Hormodendrum, Phialophora, Rhinosporidium, Microsporum, Trichophyton, Epidermophyton, Candida, Pityrosporum, and the like.

Additional agents contemplated for use in invention ligand-SDLMs and methods to induce or enhance immunity in a subject include all antigenic agents described herein as well as all nucleic acid agents encoding antigens. Such methods include administration to a subject of an amount of ligand-SDLM effective to induce or enhance an immune response (e.g., protective), whether cellular or humoral, as described herein. An effective amount is an amount effective to induce or enhance a detectable immune response. As those of skill in the art will understand, this amount will vary depending on the nature of the agent (e.g., antigen), the relative state of the subject's immune system, the age, weight and size of the subject, and the like. In vitro assays to determine whether or not a particular antigen induces an immune response are well known in the art (see Current Protocols in Immunology, 1992, Coligan et al., Wiley & Sons, eds.

In another embodiment of the present invention there are provided ligand-SDLM compositions and methods employing same for suppressing the activity of pathogenic T cells, e.g., by enhancing the activity of T suppressor cells or by inducing the production of suppressor cytokines by APC. Agents that are useful in these compositions and methods include cytokines, and the like. The method can be used to introduce any of a variety of exogenous genes into a host cell, including cytokines, chemokines, neuropeptides, islet antigens, antioxidative gene products, anti-apoptotic gene products, co-stimulatory proteins, and the like biologically active proteins and peptides. A preferable cytokine/chemokine includes interleukin (IL)-4, -5, -6, -7, -10, -13, 12-p40, TGF-β RANTES, MIF-1α or β, CCR4, CCR5 and the like molecules. Preferred neuropeptides include glucagon-like peptide-1 (GLP-1), substance P, calcitonin, and the like. Preferred islet antigens include GAD 65, insulin, ICA and the like. Preferred anti-oxidative or anti-apoptotic proteins include thioredoxin, Fas, FasL, and the like proteins. Preferred costimulatory proteins include CTLA4 Ig, CD40, CD28, B7.1, B7.2 or their ligands, and the like or antibodies that bind to any of the above molecules.

Invention ligand-SDLM compositions and methods to suppress the activity of pathogenic T cells can be employed to treat autoimmune diseases. Agents useful for such ligand-SDLM compositions and methods include, for example, GAD65/67, proinsulin, insulin B chain, heat shock proteins (e.g., HSP60), IA-2, islet cell antigens and derivatives and analogs of the forgoing, when the autoimmune disease is type I diabetes. When the autoimmune disease is multiple sclerosis, agents useful for such ligand-SDLM compositions and methods include, for example, myelin basic protein (MBP), proteolipid protein (PLP), other myelin associated proteins, and derivatives and analogs of the forgoing. When the autoimmune disease is rheumatoid arthritis, agents useful for such ligand-SDLM compositions and methods include, for example, collagen, heat shock proteins, and the like.

In still another embodiment of the present invention, there are provided ligand-SDLM compositions and methods directed to the selective killing or inhibiting the growth of neoplastic cells. Agents that may be employed in the practice of this embodiment include cytotoxic factors, cytostatic, pro-apoptotic factors, toxins and the like. Such agents may include, for example, doxorubicin, cisplatin, taxol, cyclophosphorimide, cholera toxin, and ricin A.

Any of the agents contemplated for use in the practice of the present invention may have an active component and an inactive component. For example, an antigenic polypeptide may be employed as agent. Such a polypeptide antigen may be part of a larger construct comprising, for example, an additional polypeptide, or the like. The antigenic portion of the polypeptide (that portion bearing one or more epitopes) may be part of a larger polypeptide with which it is naturally associated, or it may be part of a larger recombinant polypeptide or construct.

In embodiments for inducing or enhancing an immune response, a nucleic acid molecule can be employed as agent in ligand-SDLM compositions employed for DNA immunization methods. In this embodiment, ligand-SDLMs deliver nucleic acid-encoding antigens for presentation via MHC class I or class II pathways. DNA immunization entails the direct, in vivo administration of DNA (e.g., vector-based) that encode the production of defined microbial antigens or other desired antigens. The de novo production of these antigens in the host's own cells results in the elicitation of antibody (i.e. humoral) or cellular immune responses that may provide protection against pathogen challenge, and persist for extended periods in the absence of further immunizations. One advantage of this technology is its ability to mimic the effects of live attenuated vaccines without the safety and stability concerns often associated with the parenteral administration of live infectious agents. Because of these advantages, considerable research efforts have focused on refining in vivo delivery systems for naked DNA that result in maximal antigen production and resultant immune responses. Any polypeptide described herein as an agent for use with ligand-SDLMs can be administered by using a DNA encoding the polypeptide, or antigenic fragment thereof.

Nucleic acids delivered to cells in invention ligand-SDLM may also be employed for the introduction of therapeutic genes. Gene therapy, like DNA-based immunization, involves introduction of new genes into cells of the body, where they will be expressed to make a desired protein. However, in contrast to DNA vaccines, an immune response against the expressed gene product is not desired for gene therapy purposes. Rather, prolonged expression of the gene product is desired to augment or replace the function of a defective gene, and thus immune responses against the gene product may be undesirable.

As used herein, “nucleic acid” or “polynucleotide” means both DNA and RNA in all of their forms. Nucleic acids contemplated for use in the practice of the present invention include naked DNA, naked RNA, naked plasmid DNA, either supercoiled or linear, and encapsulated DNA or RNA (e.g., in liposomes, microspheres, or the like). As will be understood by those of skill in the art, particles mixed with plasmid so as to “condense” the DNA molecule may also be employed. Delivery of polynucleotides can be achieved using a recombinant expression vector such as a chimeric virus, or the polynucleotide can be delivered as “naked” DNA in an ligand-SDLM, for example.

As those of skill in the art readily appreciate, nucleic acids may be introduced into a cell for a variety of purposes, including, introduction of exogenous expressible genes, introduction of nucleic acid for the disruption of native gene expression (e.g., introduction of antisense nucleic acid, ribozymes), and the like. Nucleic acids contemplated for use in the practice of the present invention can be double stranded DNA (e.g., plasmid, cosmid, phage, viral, YACS, BACS, other artficial chromsomes, and the like), single stranded DNA or RNA. The nucleic acids may be uncomplexed (i.e., “naked”) or complexed (e.g., with chemical agents such as lipids (e.g., cationic), dendrimers, or other polyplexes that facilitate DNA penetration into tissues and through cell membranes, and the like). The DNA may also be encapsulated or formulated with protein complexes, so long as it is able to be complexed with ligand for binding to a CSR and subsequent uptake by a cell.

DNA and mRNA can be delivered to cells where they express a polypeptide translation product. When the nucleic acids are operatively associated with the proper regulatory sequences, enhanced synthesis of the encoded protein is achievable. DNA or RNA encoded polypeptides contemplated for use in the practice of the present invention include immunizing polypeptides, pathogen-derived proteins, blood coagulation factors, peptide hormones, and the like. Peptide hormones include, for example, calcitonin (CT), parathyroid hormone (PTH), erythropoietin (EPO), insulin, lymphokines, cytokines, growth hormone, growth factors, and the like. Lymphokines and cytokines contemplated for use in the practice of the present invention include those described above with reference to certain other embodiments of the present invention, including, for example, tumor necrosis factor, interleukins 1, 2, and 3, lymphotoxin, macrophage activating factor, migration inhibition factor, colony stimulating factor, alpha-interferon, beta-interferon, gamma-interferon and subtypes thereof.

Invention methods can be applied to achieve improved and more effective immunity against infectious agents, including bacteria, intracellular viruses, tumor cells, and the like by delivering DNA or mRNA. Therapeutic polynucleotides provided by the invention can also code for immunity-conferring polypeptides, which can act as endogenous immunogens (e.g., antigen-containing polypeptides) to provoke a humoral immune response, a cellular immune response, or both. Methods for inducing such responses and targeting specific cells for specific responses are described, for example, in U.S. Pat. No. 5,589,466.

The polynucleotides employed in accordance with the present invention can also encode an antibody. The term “antibody” encompasses whole immunoglobulin of any class, anti-idiotype antibodies, chimeric antibodies and hybrid antibodies with dual or multiple antigen or epitope specificities, and fragments, such as F(ab)₂, Fab′, Fab, and the like, including hybrid fragments thereof. Also included within the meaning of “antibody” are conjugates of such fragments, and so-called antigen binding proteins (single chain antibodies) as described, for example, in U.S. Pat. No. 4,704,692, hereby incorporated by reference.

Thus, an isolated polynucleotide coding for variable regions of an antibody can be introduced, in accordance with the present invention, to stimulate a subject's immune system to produce antibody in situ. For illustrative methodology relating to obtaining antibody-encoding polynucleotides, see Ward et al. Nature, 341:544-546 (1989); Gillies et al., Biotechnol. 7:799-804 (1989). The antibody in turn may exert a therapeutic effect, for example, by binding a surface antigen associated with a pathogen. Alternatively, encoded antibodies can be anti-idiotypic antibodies (antibodies that bind other antibodies) as described, for example, in U.S. Pat. No. 4,699,880. Such anti-idiotypic antibodies may bind endogenous or foreign antibodies in a treated individual, thereby ameliorating or preventing pathological conditions associated with an immune response, (e.g., in the context of an autoimmune disease such as lupus, arthritis, and the like).

Polynucleotide sequences used in the practice of the present invention encode therapeutic or immunogenic polypeptides. These polynucleotide sequences may be used in association with other polynucleotide sequences coding for regulatory proteins that control the expression of the therapeutic or immunogenic polypeptides. The regulatory protein(s) so employed can act in any number of regulatory manners known to those of skill in the art, such as by binding to DNA so as to regulate its transcription, by binding to messenger RNA to increase or decrease its stability or translation efficiency, and the like.

The polynucleotide material delivered to the cells in vivo can take any number of forms, and the present invention is not limited to any particular polynucleotide coding for any particular polypeptide. Plasmids containing genes coding for a large number of physiologically active peptides and antigens or immunogens are contemplated for use in the practice of the present invention and can be readily obtained by those of skill in the art.

Various viral vectors can also be utilized in the practice of the present invention and include adenovirus, herpes virus, vaccinia, RNA virus, and the like. It is presently preferred that the virus be an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). When the subject is a human, a vector such as the gibbon ape leukemia virus (GaLV), or the like can be utilized. A number of additional retroviral vectors can incorporate multiple genes. All of these vectors can transfer or incorporate a gene for a selectable marker so that transduced cells can be identified and generated.

In addition to the viral vectors described herein, plasmid DNA expression vectors are also useful as agents for gene therapy embodiments. They may be preferable to viral vectors (i.e., recombinant adenovirus or retrovirus), which themselves are immunogenic (Newman et al., 1995; Zabner et al., 1996). Immune responses directed against such vectors may interfere with successful gene transfer if the same vector is used more than once. Double-stranded DNA is not immunogenic, and thus from this perspective, repeated use is not a problem with plasmid DNA.

As will be understood by those of skill in the art, efficient expression of a nucleic acid encoding either a therapeutic or antigenic polypeptide generally requires that the nucleic acid sequence be operably associated with a regulatory sequence. Regulatory sequences contemplated for use in the practice of the present invention include promoters, enhancers, and the like. As those of skill in the art will also appreciate, even when a promoter sequence is operably associated with the therapeutic nucleic acid, expression may be further augmented by operably associating an enhancer element or the like. Promoters contemplated for use in DNA vaccines of the present invention include the CMV, RSV LTR, MPSV LTR, SV40, tissue specific promoters (e.g., the keratin and involucrin group of promoters), and the like.

In addition to the general classes of polypeptide and nucleic acid agents described herein, therapeutic agents may be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs. Candidate agents are also found among biomolecules including, but not limited to: peptides, saccharides, fatty acids, steroids, purines, pyrimidines, derivatives, structural analogs or combinations thereof.

The invention will now be described in greater detail by reference to the following non-limiting examples.

### EXAMPLES

### Example 1

### In Vivo Internalization of SDLM Engineered with Different Concentrations of Ligand

| Ligand (hIgG) | Percentage range of |
|---|---|
| concentration in | bronchoalveolar lavage cells |
| SDLM | positive for Texas Red* |
| No ligand (n = 6) | <0.1% (n = 6 rats) |
| Ligand content | 1-30% (n = 10 rats) |
| (50%) | |

*Data based on microscopy and FACS analysis.

SDLM comprising 25% of polyclonal human IgG (hIgG), 10% HES (hydroxyethyl starch), and 65% lipid (DPPC=dipalmitoyl phosphotidyl choline/DSPC=distearylphosphatidyl choline; 1% of lipid covalently tagged with Texas Red) were administered as dry powder to anesthetized Sprague Dawley rats, via the intratracheal route (percents are by weight). The dose (1 mg/kg) was administered intratracheally using an insufflator device (Insufflator™, Penn-Century). At one hour after the administration, the bronchoalveolar cells were harvested by standard saline lavage of the lungs. The cells were placed on ice for all subsequent manipulations. After washing with 4° C. phophate-buffered saline (PBS), the cells were cytospun onto slides and counterstained with a nuclear stain (SYTO-16) for 30 minutes in 1×PBS. Fluorescent microscopy was carried out using an epifluorescence microscope (Jena-Sedival) equipped with video imaging and digital enhancement software (Image-Pro Analysis). Internalization of microparticles was observed.

Similar protocols were carried out using SDLM that were formulated with or without (blank controls) 50% human IgG. The quantitation of cells that incorporated Texas Red-tagged SDLM was carried out by both fluorescent microscopy and FACS analysis (Coulter) after gating the SYTO-16⁺ events. Less than 1/1000 cells were positive for Texas Red subsequent to administration of non-ligand-containing SDLM. In contrast, most of the animals treated with ligand-engineered SDLM displayed a frequency of Texas Red⁺ cells that was two orders of magnitude higher (range: 1/100 to ⅓).

In conclusion, whereas non-ligand engineered SDLM were not taken up, ligand engineered SDLM were internalized by airway phagocytes.

Spray dried ligand based microparticles are constructed according to a method previously described in the art (WO99/16420; PCT US99/06855). Briefly, an emulsion of perfluorocorbon by water is obtained and stabilized using lipids with surfactant properties (like DPPC/DSPC). A solution comprising ligand, agent and other excipients (e.g, carbohydrates, salts) is added to the emulsion. The mixture is spray-dried and dessicated.

### Example 2

### Ligand Dependency of SDLM Internalization into Phagocytic Cells

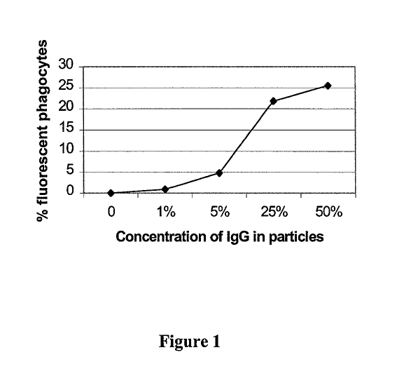

Bronchoalveolar lavage (BAL) cells were harvested from anesthetized Sprague Dawley rats using normal saline and washed with serum-free medium. They were incubated for 1 hour at 37° C. in 5% CO₂ in 24-well plates at a concentration of 2×10⁵ cells/well. After the incubation, the non-adherent cells were gently washed off and SDLM comprising 65% lipid (DPPC/DSPC) (100 μg/well), freshly resuspended in normal saline, were added to the adherent cells supplemented with cell culture medium. Particles containing 10% HES, with different concentrations of hIgG (0%, 1%, 5%, 10%, 25% and 50%) were used. After another 1-hour incubation at 37° C. in 5% CO₂, the cells were washed with PBS three times and counterstained with SYTO-16 (nuclear dye) for 30 minutes at room temperature. After another wash with PBS, uptake was assessed by fluorescence microscopy, using an epifluorescence microscope (Jena-Sedival) equipped with video imaging and digital enhancement software (Image-Pro Analysis). More than 500 cells/well were analyzed. The results are depicted in FIG. 1 and were independently confirmed by dual-color flow cytometry (Coulter), after gating for SYTO-16⁺ events, which eliminates any noise due to lipid aggregates. These results indicate that the internalization of SDLM is dependent on the amount of co-formulated ligand (in this case IgG ligand).

### Example 3

### Both Lipid Matrix and Ligand are Co-internalized into Phagocytes

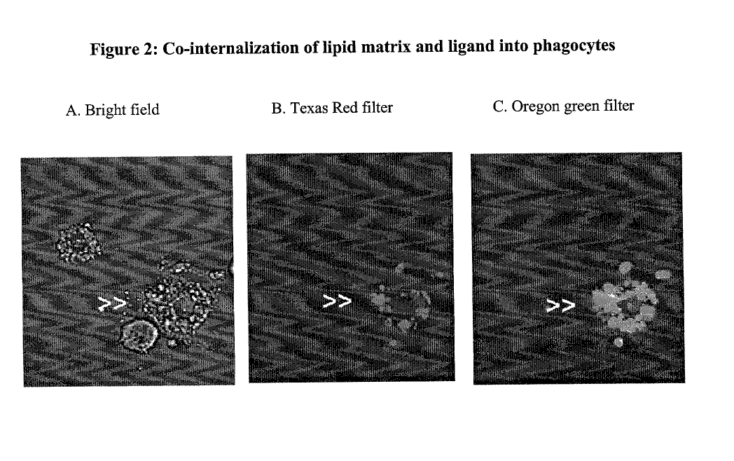

Bronchoalveolar lavage cells were harvested from anesthetized Sprague Dawley rats and adherent cell samples prepared as described in Example 2 above. SDLM engineered with Oregon Green-tagged hIgG (100 μg formulation/well), which had been freshly resuspended in normal saline, were added to the adherent cells supplemented with serum free medium. The SDLM were composed of 10% HES, 25% tagged hIgG and DPPC/DSPC tagged with Texas Red. After another 1 hour incubation at 37° C. in 5% CO₂, the cells were washed with PBS three times and uptake assessed by fluorescence microscopy, using a microscope (Jena-Sedival) equipped with video imaging and digital enhancement software (Image-Pro Analysis). More than 500 cells/well were analyzed. As shown in the FIG. 2, significant co-internalization of tagged molecules into bronchoalveolar lavage cells was observed, which indicates that this process is mediated by ligand-receptor interaction.

### Example 4

### Inefficiency of Opsonization in Promoting Internalization of Non-ligand Engineered Microparticles

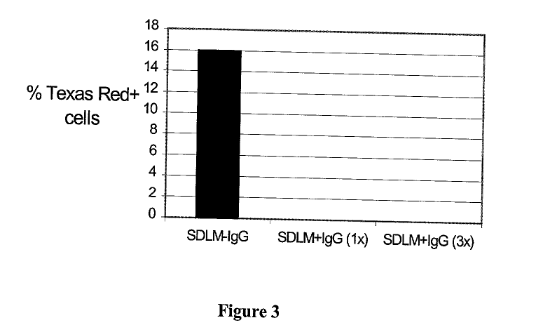

Bronchoalveolar lavage cells were harvested from anesthetized Sprague Dawley rats and adherent cell sampes prepared as described in foregoing Examples. SDLM (100 μg formulation/well), freshly resuspended in normal saline, were added to the adherent cell samples supplemented with cell culture medium. The SDLM were composed of 10% HES and DPPC/DSPC tagged with Texas Red either with (FIG. 3, first column) or without (FIG. 3, second and third columns) 25% hIgG ligand. The incubation of the non-ligand containing SDLM was carried out in the presence of 25 μg hIgG (FIG. 3, second column, 1×-molar ratio relative to amount of formulated IgG corresponding to the first column) or 75 μg hIgG (third column, 3×molar ratio relative to amount of formulated IgG corresponding to the first column) added as an aqeuous saline solution. After another 1 hour incubation at 37° C. in 5% CO₂, the cells were washed with PBS three times and counterstained with SYTO-16. Uptake was quantified by fluorescence microscopy, using a microscope (Jena-Sedival) equipped with video imaging and digital enhancement software (Image-Pro Analysis). The number of Texas Red⁺ cells in the last two columns was less than 0.1%. Thus, the hIgG internalization effect is only present when the hIgG is formulated as ligand within the SDLM, not in free media alone.

### Example 5

### Inhibition of Receptor-mediated Internalization of Ligand-SDLM by Soluble Ligand in Excess

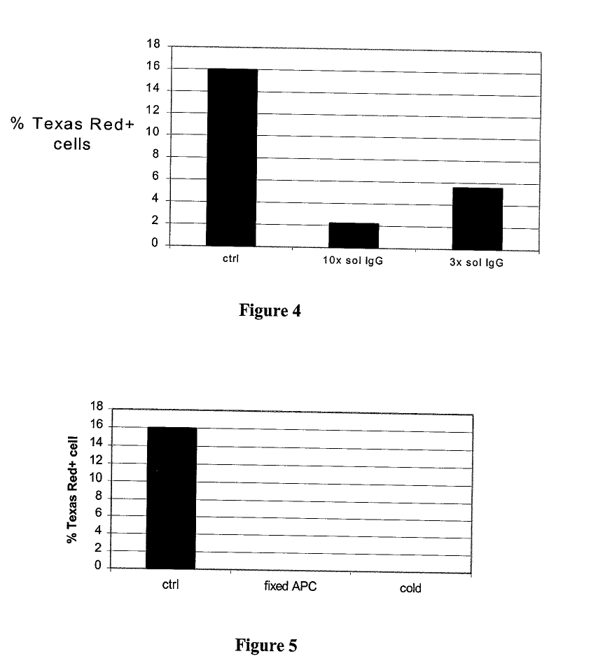

Bronchoalveolar lavage cells were harvested from anesthetized Sprague Dawley rats and processed as described in the above Examples. SDLM (100 μg formulation/well), freshly resuspended in normal saline, were added to the adherent cell samples supplemented with serum-free medium. The SDLM comprised 10% HES, 25% hIgG and DPPC/DSPC tagged with Texas Red (‘ctrl’). In parallel wells, the incubation of ligand-SDLM was carried out in the presence of 250 μg (FIG. 4, second column, ‘10×’-molar ratio relative to amount of formulated IgG) or 75 μg (FIG. 4, third column, ‘3×’-molar ratio relative to amount of formulated IgG) of hIgG added as solution in normal saline. After another 1-hour incubation at 37° C. and 5% CO₂, the cells were washed with PBS three times and counterstained with SYTO-16. The results were acquired by fluorescent microscopy, using a system (Jena-Sedival) equipped with video imaging and digital enhancement software (Image-Pro Analysis). These results shown in the FIG. 4 confirm that an IgG receptor mediates the internalization of SDLM with IgG ligand, as excess free IgG was able to compete off SDLM for binding to bronchoalveolar lavage cells.

### Example 6

### Cellular Internalization of Ligand-SDLM is an Active Process

Bronchoalveolar lavage cells were harvested from anesthetized Sprague Dawley rats and processed as described in Examples above. SDLM composed of 10% HES and DPPC/DSPC tagged with Texas Red, and 25% hIgG, were added to the adherent cell samples (100 μg formulation/well). In this experiment, three different categories of adherent cells were used: first, live cells (APC) at 37° C. and 5% CO₂; second, cells that had been previously fixed with 1% paraformaldehyde; and third, live cells kept at 4° C. throughout the incubation step. After incubation, the cells were washed with PBS three times and counterstained with SYTO-16. The results were acquired by fluorescence microscopy, using a microscope (Jena-Sedival) equipped with video imaging and digital enhancement software (Image-Pro Analysis). More than 500 cells/well were analyzed. The results are depicted in FIG. 5, and indicate that the internalization is an active rather than passive (e.g., diffusion mediated) process, consistent with ligand-receptor mediated phagocytosis.

### Example 7

### Effect of Carbohydrate Excipients and Microparticle Stability on Receptor-mediated Internalization of Ligand-SDLM

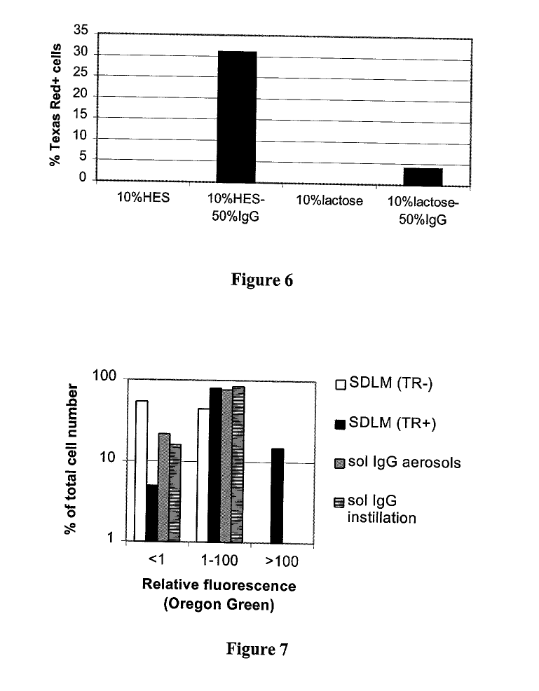

Bronchoalveolar lavage cells were harvested from anesthetized Sprague Dawley rats and plastic-adherent cells prepared as described in the Examples above. SDLM (100 μg formulation/well), freshly resuspended in normal saline, were added to the adherent cells supplemented with serum-free medium. The formulations used included: SDLM with 10% HES (high molecular weight carbohydrate, associated with increased particle stability) or with 10% lactose (low molecular weight carbohydrate, associated with decreased stability of particles in saline). In each case, non-ligand engineered and ligand engineered SDLM (50% hIgG) were tested. Texas Red-tagged DPPC (0.1%) was incorporated as the indicator. After the incubation, the cells were washed with PBS three times and counterstained with SYTO-16. The results were acquired by fluorescence microscopy, using a microscope (Jena-Sedival) equipped with video imaging and digital enhancement software (Image-Pro Analysis). More than 500 cells/well were analyzed. The results are depicted in FIG. 6, and indicate that HES is an effective stability conferring excipient as compared to a low molecular weight carbohydrate (lactose).

### Example 8

### Enhanced Uptake of Ligand-SDLM into Bronchoalveolar Phagocytes In Vivo

SDLM comprising 25% of polyclonal human IgG (hIgG) tagged with Oregon Green, 10% HES and 65% lipid (DPPC /DSPC; 1% of DPPC covalently tagged with Texas Red) were administered as dry powder to anesthetized Sprague Dawley rats, via the intratracheal route. The dose (1 mg/kg) was administered intratracheally using an insufflator device (Insufflator™, Penn-Century). As a control, three rats/group received dose-matched tagged hIgG in saline via aerosols or liquid instillation into the trachea. At one hour after the administration, the bronchoalveolar cells were harvested by standard wash with normal saline. The cells were manipulated and transported on ice. After washing with 4° C. PBS, uptake was determined using dual-color flow cytometry (Coulter). The cellular internalization of tagged hIgG (ligand) following administration of engineered SDLM was quantified by gating for Texas Red+ versus Texas Red− cells. High levels of internalized hIgG were noted only in cells that incorporated SDLM, from rats treated with ligand-engineered particles. The results depicted in FIG. 7 are representative for n=3/group and demonstrate increased internalization of ligand in a subset of phagocytes targeted with ligand-SDLM.

### Example 9

### Incorporation of Viral Antigen (UV-inactivated A/WSN/32 H1N1 Influenza Virus) into Ligand-SDLM and Release-kinetics of Antigen Versus Ligand

Microparticles containing 5% UV-inactivated influenza virus (A/WSN/32 H1N1) and 10% mouse monoclonal IgG2b were obtained by spray-drying, using as excipients a mixture of DPPC and DSPC, as well as 10% HES. The composition of this prototype antigen, ligand-engineered formulation are shown in the Table (I) below:

Composition of ligand-SDLM

| Components | Composition |
|---|---|
| Excipients: | |
| Phosphatidylcholine | 75% |
| (DSPC + DPPC) | |
| Hydroxyethylstarch | 10% |
| Ligand: | |
| IgG_{2b} | 10% |
| Active agent: | |
| Virus antigen^{a} | 5% |
| Infectious virus^{b} | <1 TCID₅₀/100 μg |

^{a}TcH assay using SDS-treated SDLM.
^{b}MDCK assay.

The amount of viral antigen was quantified after dissolution of lipid particles with saline supplemented with 0.1% SDS for 30 minutes at room temperature (vigorous shaking). After the removal of SDS using two rounds of depletion on detergent-removal columns (SDS-OUT; Pierce), the concentration of viral antigen was estimated by bioassay (incubation with reporter-engineered 16-2-6 T cell hybridoma specific for the dominant epitope HA 110-120, in the presence of M12 antigen presenting cells). For the standard curve, detergent-treated UV inactivated WSN virus was used. The estimation of infectious viral titre was carried out by standard titration on MDCK cells: after 48-hour incubation of SDLM on permissive cells, the supernatant was tested for the presence of influenza virus by hemagglutination. The concentration of ligand (mouse IgG2b k, MOPC 141) was confirmed by ELISA after detergent dissolution of particles, using polyclonal goat anti-mouse IgG antibodies and a standard curve constructed with MOPC 141. The estimation of endotoxin was carried out using standard Limulus assay (Bio-Whittaker) and showed less than 0.5EU/20 mg of formulation.

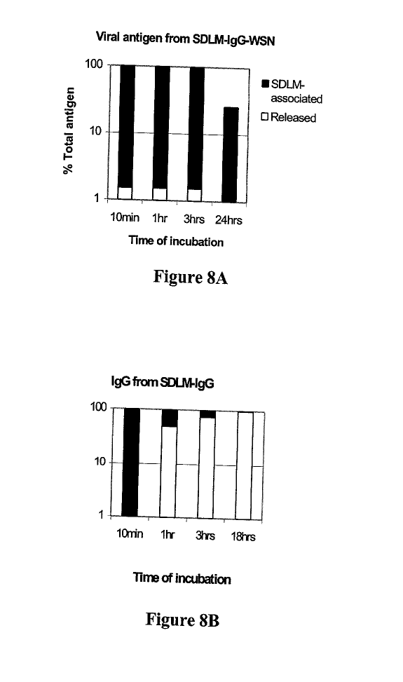

Using a similar strategy to estimating the amount of viral antigen (bioassay with specific T cell hybridoma—16-2-6 TcH and M12 antigen presenting cells), the release-kinetics of antigen from ligand-engineered SDLM were also estimated (FIG. 8A) as follows. The particles were suspended in normal saline at 37° C. under mild shaking. Aliquots were harvested at various intervals and centrifuged for 5 minutes at 10,000 RPM. The amount of antigen was quantified in the supernatants. In the case of IgG-SDLM (FIG. 8B) the concentration of immunoglobulin in the supernatant was measured by ELISA.

The data demonstrate that both ligand and antigen are retained for a certain time window in SDLM exposed to an aqueous environment.

### Example 10

### Presentation of an MHC Class II-restricted Dominant Epitope (HA 110-120) Form the Context of Ligand-SDLM by Bronchoalveolar Phagocytes

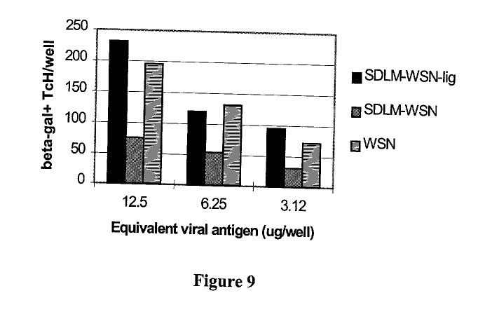

Airway APC were isolated from BALB/c mice by standard bronchoalveolar lavage using normal PBS. The recovered cells were washed with 4° C.-cold cell culture medium (HL-1) twice and incubated in 96-well flat-bottom plates (1×10⁵ cells/well) with various amounts of dried-SDLM, corresponding to defined quantities of viral antigen (as shown in FIG. 9). After 1 hour incubation at 37° C. under mild horizontal shaking conditions (30 rpm), the non-adherent cells and lipid debris were washed off by repeated, gentle addition and removal of HL-1 medium. T cell hybridoma (16-2-6) specific for HA 110-120 epitope of WSN virus were added to the plastic-adherent cells (2×10⁴ TcH/well in 100 μl of HL-1 medium). After 12-hour incubation at 37° C. and 5% CO₂, the cells were fixed with glutaraldehyde/formaldehyde and X-gal substrate was added. The results in FIG. 9 are expressed as the number of activated TcH/well. The data shown are the average of duplicate experiments. In conclusion, addition of a ligand to SDLM improved the efficiency of antigen presentation by bronchoalveolar phagocytes, as compared to non-ligand engineered SDLM loaded with antigen.

### Example 11

### Presentation of an MHC Class II-restricted Dominant Epitope (HA 110-120) Form the Context of Ligand-SDLM by Lung-derived Antigen Presenting Cells

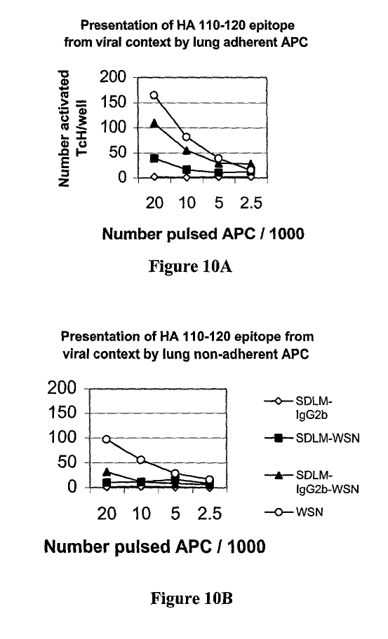

Lungs from naïve BALB/c mice were mildly digested with collagenase (60 minutes at 37° C. in the presence of 30 U/ml of collagenase from C. histolyticum) and passed through 70 μm strainers. After hypotonic lysis of erythrocytes, the resulting cell suspension was incubated on plastic wells for 2.5 hours at 37° C. and 5% CO₂ at a concentration of 2×10⁶ cells/2 ml of serum-free HL-1 medium, in the presence of microparticles loaded with inactivated virus (corresponding to 50 μg of antigen/well) or dose-matched, non-formulated virus. Non-adherent cells were harvested by gentle washing with cell culture medium at room temperature and adherent cells were harvested by forceful pipetting with 4° C. PBS, after a 15-minute incubation on ice. The cells were washed and various numbers incubated with 2×10⁴/well 16-2-6 TcH overnight at 37° C. and 5% CO₂. The cells were fixed and X-gal substrate was added. The quantification of activated TcH was carried out by microscopy. The results were expressed as number of activated TcH depending on the number of pulsed adherent (FIG. 10A), or non-adherent (FIG. 10B) antigen presenting cells.

By flow-cytometry, approximately 20% or 50% of non-adherent or adherent cells, respectively, expressed MHC class-II molecules. SDLM-WSN (particles loaded with 5% UV-inactivated virus; SDLM-IgG2b-WSN are particles loaded with the same amount of virus and engineered with 10% IgG2b ligand; WSN represents soluble, dose-matched killed virus; and SDLM-IgG2b are control ligand-engineered particles devoid of viral antigen).

Taken together, the data show that co-formulation of IgG ligand resulted in increased presentation of antigen by adherent APC isolated from lung, as compared to non-ligand engineered SDLM.

### Example 12

### Effect of Ligand-engineering of SDLM on the Presentation of Virus Epitopes to Specific T Cells by Splenic Antigen Presenting Cells

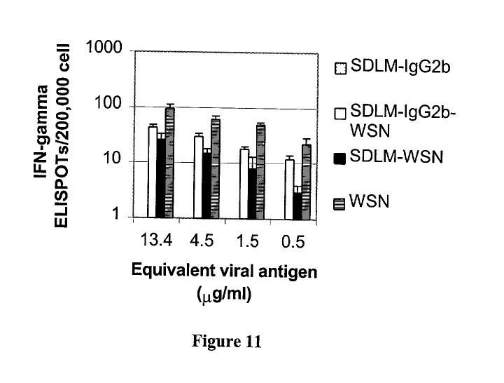

Splenocytes were obtained from BALB/c mice previously immunized with live WSN virus (intraperitoneally, with 1×10⁵ TCID₅₀) by mincing splenic tissue and passing the resulting material through 70 mm strainers. After standard hypotonic lysis of erythrocytes, the splenocytes were washed and incubated in anti-IFNγ or anti-IL-4 coated ELISPOT plates at 2×10⁵ cells/well, in HL-1 medium. Various amounts of SDLM freshly resuspended in HL-1 medium were added to the splenocytes. After incubation at 37° C. in 5% CO₂ for 72 hours, the cells were washed off and either biotinylated anti-IFNγ or anti-IL-4 was added (overnight incubation at 4° C. in PBS-2% FCS supplemented with 0.05% Tween 20). After extensive washing, the ELISPOT plates were developed using streptavidin-HRP conjugate followed by insoluble peroxidase substrate (AEC). The number of cytokine producing colonies per well was quantified with a magnifying glass. The experiment was carried out in triplicate wells. The results are shown in FIG. 11 as mean±SE spots/2×10⁵ splenocytes. No expansion of IL-4 producing T cell colonies was noted by restimulation with SDLM in the above mentioned conditions. The background (no antigen) was consistently between 0-2 spots/well. As additional controls, SDLM was used with ligand but devoid of virus antigen (SDLM-IgG2b) or dose-matched, soluble (innactivated) WSN virus.

The data show that ligand engineering of SDLM improved the antigen presentation of virus, as compared to non-ligand engineered microparticles.

### Example 13

### Dependency of Antigen-processing and Epitope Presentation From the Context of Ligand-SDLM on Particle Internalization Subsequent to Ligand-receptor Interaction

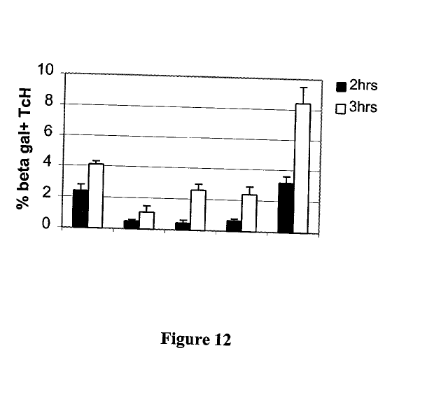

The antigen presenting cells used in this example were bronchoalveolar (BA) phagocytes isolated by standard bronchoalveolar saline lavage of BALB/c mice. After washing with 4° C.-cell culture medium, the cells were incubated in plastic wells for 60 minutes at 37° C. in 5% CO₂, at a concentration of 1×10⁶ cells/ml in 2 ml. The non-adherent cells were gently washed off and the adherent cells were detached by forceful pipetting following 15-minute incubation at 4° C. A subset of the BA cells was coated with mouse polyclonal IgG for 60 minutes at 4° C. (50 times molar excess relative to the amount of hIgG ligand in the SDLM added subsequently). Non-coated and coated BA cells were pulsed with ligand-engineered particles containing virus antigen, or with dose-matched virus (4 μg viral antigen/2×10⁴ BA cells/well), for 60 minutes at 37° C. and 5% CO₂. The cells were gently washed three times with HL-1 medium and 2×10⁴ specific TcH were added in each well. After 2 or 3-hour incubation at 37° C. and 5% CO₂, the cells were fixed with glutaraldehyde+formaldehyde and X-gal was added. The results depicted in FIG. 12 are expressed as mean±SE of percentage activated (galactosidase⁺) TcH. As control, M12 B lymphoma APC pulsed with soluble inactivated virus is shown in the last set of columns.

These data demonstrate that the interaction between ligand (IgG) and receptor (FcR) is a prerequisite for antigen presentation from the context of ligand-engineered SDLM, since it can be significantly inhibited by soluble ligand in excess. In contrast, as expected, the presentation of non-formulated virus was not inhibited by soluble ligand (IgG), since it involves engagement of sialoreceptors by viral hemagglutinin. While the invention has been described in detail with reference to certain preferred embodiments thereof, it will be understood that modifications and variations are within the spirit and scope of that which is described and claimed.

We claim:

1. A Spray-Dried Lipid Microparticle (SDLM) composition, comprising one or more phospholipids, a therapeutic or biologically active agent, and at least one ligand that binds to a cell surface receptor.

2. A composition according to claim 1, wherein the cell surface receptor is coupled with a signal cascade leading to endocytosis/phagocytosis of the receptor upon binding of ligand to receptor.

3. A composition according to claim 1, wherein the cell surface receptor is selected from the group consisting of FcR, mannose receptor, scavenger receptor, viral receptor, M-cell receptor, ganglioside GM1 receptor, transferrin receptor, complement receptors, surface immunoglobulins on B cells, T cell receptors on T cells, peptidoglycan receptor, LPS receptor, and glycolipid receptor.

4. A composition according to claim 1, wherein the ligand comprises an Fc portion of an antibody.

5. A composition according to claim 1, wherein the ligand binds to a cell surface receptor selected from the group consisting of FcR, mannose receptor, scavenger receptor, viral receptor, M-cell receptor, ganglioside GM1 receptor, and transferrin receptor.

6. A composition according to claim 1, wherein the agent comprises an antigen.

7. A composition according to claim 6, wherein the antigen is a polypeptide or a whole-inactivated pathogen.

8. A composition according to claim 7, wherein the antigen is a pathogen-derived polypeptide.

9. A composition according to claim 8, wherein the pathogenic antigen or whole inactivated pathogen is selected from the group consisting of a viral antigen, a bacterial antigen, a yeast antigen, a fungal antigen, and a protozoan antigen.

10. A composition according to claim 9, wherein the bacterial antigen is derived from, or the whole-inactivated pathogen is a bacterium selected from the group consisting of Staphylococcus, Clostridium, Streptococcus, Enterococcus, Diplococcus, Hemophilus, Neisseria, Erysipelothricosis, Identifyeria, Bacillus, Salmonella, Shigella, Escherichia, Klebsiella, Enterobacter, Serratia, Proteus, Morganella, Providencia, Yersinia, Camphylobacter, and Mycobacteria.

11. A composition according to claim 9, wherein the fungal antigen is derived from, or the whole-inactivated pathogen is a fungus selected from the group consisting of Histoplasma, Coccidioides, Cryptococcus, Blastocyces, Paracoccidioides, Candida, Aspergillus, Nocardia, Sporothrix, Rhizopus, Absidia, Mucor, Hormodendrum, Phialophora, Rhinosporidium, Microsporum, Trichophyton, Epidermophyton, Candida, and Pityrosporum.

12. A composition according to claim 9, wherein the protozoan antigen is derived from, or the whole-inactivated pathogen is a protozoa selected from the group consisting of Plasmodium, Trypanosoma, Microfilariae, Leishmania, Giardia, Entamoeba, Schistosoma, Cryptosporidium Entamoeba, and Pneumocystis.

13. A composition according to claim 9, wherein the viral antigen is derived from, or the whole-inactivated pathogen is a virus selected from the group consisting of influenza virus (orthomyxoviruses), papiloma virus, herpes simplex virus, Epstein Barr virus, measles virus, rhinovirus, poliovirus, rotavirus, varicella-virus, and HIV.

14. A composition according to claim 7, wherein the polypeptide is a tumor cell-derived polypeptide.

15. A composition according to claim 7, wherein the polypeptide is recombinant.

16. A composition according to claim 15, wherein the recombinant polypeptide comprises at least one epitope that is recognized by an autoimmune response.

17. A composition according to claim 16, wherein the epitope is derived from a polypeptide selected from the group consisting of GAD65/67, proinsulin, insulin B chain, a heat shock protein, IA-2, an islet cell antigen, myelin basic protein, myelin proteolipid protein, myelin oligodendrocyte glycoprotein; myelin P2 protein; acethylcoline receptor and collagen.

18. A composition according to claim 17, wherein the heat shock protein is selected from the group consisting of HSP 15, HSP 32, HSP 60, HSP 65, HSP 70, HSP 72, HSP 73, and HSP 90.

19. A composition according to claim 1, wherein the therapeutic agent is selected from the group consisting of a cytotoxic/cytostatic factor, a pro-apoptotic factor, and a toxin.

20. A composition according to claim 19, wherein the cytotoxic/cytostatic factor is selected from the group consisting of doxorubicin, cytarabin, paclitaxel, epoxid-piperazines, 5-fluorouracil, melphalan, vincristine, and cyclophosphamide.

21. A composition according to claim 19, wherein the pro-apoptotic factor is selected from the group consisting of ganciclovir, penciclovir, indonocine, and FasL.

22. A composition according to claim 19, wherein the toxin is selected from the group consisting of cholera toxin, pertussis toxin, and ricin A toxin.

23. A composition according to claim 1, wherein the phospholipid comprises in the range of about 25 to 90% by weight of the SDLM.

24. A composition according to claim 1, wherein the phospholipid comprises about 65% by weight of the SDLM.

25. A composition according to claim 1, further comprising a stability-conferring excipient.

26. A composition according to claim 25, wherein the stability-conferring excipient is a carbohydrate or a salt based on a divalent cation.

27. A composition according to claim 26, wherein the carbohydrate is selected from the group consisting of a monosaccharide, a polymeric straight chain carbohydrate and a branched chain polysaccharide.

28. A composition according to claim 27, wherein said polymeric straight or branched polysaccharide is selected from the group consisting of hydroxyethyl starch (HES), mannan, and laminarin.

29. A composition according to claim 27, wherein said carbohydrate is lactose.

30. A composition according to claim 1, wherein the phospholipid is phosphatidylcholine or a derivative thereof selected from the group consisting of dipalmitoylphosphatidylcholine; dystearylphosphatidylcholine; dimiristoylphosphatidylcholine and combinations of two or more thereof.

31. A composition according to claim 1 comprising about 65% phosphatidylcholine or a derivative thereof, about 10% high molecular weight carbohydrate, in the range of about 10-20% IgG ligand, and in the range of about 5-15% antigen.

32. A composition according to claim 1, wherein the SDLM is hollow.

33. A composition according to claim 1, wherein the SDLM is porous.

34. A composition according to claim 1, wherein the SDLM is non-porous.

35. A pharmaceutical composition comprising an SDLM of claim 1 in a pharmaceutically acceptable carrier.

36. A method for the introduction of a therapeutic or biologically active agent into a cell of a subject, comprising administering to a subject a composition according to claim 1.

37. A method according to claim 36, wherein the ligand and agent are coupled such that upon to binding of the ligand to the cell surface receptor, a ligand-agent-receptor complex is formed and subsequently is internalized by the cell, thereby resulting in introduction of the agent into the cell.

38. A method according to claim 36, wherein the cell is a macrophage.

39. A method according to claim 36, wherein the cell is an antigen presenting cell (APC).

40. A method according to claim 36, wherein the therapeutic agent is an antigen.

41. A method according to claim 37, wherein the internalization of the antigen induces an immune response in the subject.

42. The method according to claim 40, wherein the antigen is a polypeptide or a whole-inactivated pathogen.

43. The method according to claim 42, wherein the polypeptide is a pathogen-derived polypeptide.

44. The method according to claim 43, wherein pathogenic antigen is selected from the group consisting of a viral antigen, a bacterial antigen, a yeast antigen, a fungal antigen, and a protozoan antigen.

45. The method according to claim 44, wherein the bacterial antigen is derived from, or the whole-inactivated pathogen is a bacterium selected from the group consisting of Staphylococcus, Clostridium, Streptococcus, Enterococcus, Diplococcus, Hemophilus, Neisseria, Erysipelothricosis, Identifyeria, Bacillus, Salmonella, Shigella, Escherichia, Klebsiella, Enterobacter, Serratia, Proteus, Morganella, Providencia, Yersinia, Camphylobacter, and Mycobacteria.

46. The method according to claim 44, wherein the fungal antigen is derived from, or the whole-inactivated pathogen is a fungus selected from the group consisting of Histoplasma, Coccidioides, Cryptococcus, Blastocyces, Paracoccidioides, Candida, Aspergillus, Nocardia, Sporothrix, Rhizopus, Absidia, Mucor, Hormodendrum, Phialophora, Rhinosporidium, Microsporum, Trichophyton, Epidermophyton, Candida, and Pityrosporum.

47. The method according to claim 44, wherein the protozoan antigen is derived from, or the whole-inactivated pathogen is a protazoan selected from the group consisting of Plasmodium, Trypanosoma, Microfilariae, Leishmania, Giardia, Entamoeba, Schistosoma, Cryptosporidium Entamoeba, and Pneumocystis.

48. The method according to claim 44, wherein the viral antigen is derived from, or the whole-inactivated pathogen is a virus selected from the group consisting of influenza virus (orthomyxoviruses), papiloma virus, herpes simplex virus, Epstein Barr virus, measles virus, rhinovirus, poliovirus, rotavirus, varicella-virus, and HIV.

49. The method according to claim 42, wherein the polypeptide is a tumor cell-derived polypeptide.

50. The method according to claim 42, wherein the polypeptide is recombinant.

51. The method according to claim 36, wherein the ligand is an immunoglobulin selected from the group consisting of IgG, IgM, IgA, IgE, and IgD.

52. The method according to claim 41, wherein the immune response is a Class I or Class II MHC-mediated response.

53. The method according to claim 52, wherein the class I MHC-mediated response is a CD8+ cytotoxic lymphocyte (CTL) response.

54. A method according to claim 37, wherein introduction of the agent results in suppression of pathogenic T cells.
